(19) United States
(12) Patent Application Publication
(10) Pub. No.: US 2019/0307617 A1
(43) Pub. Date: Oct. 10, 2019

(54) THREE-DIMENSIONAL SHAPED FORMS FOR USE IN DISPOSABLE ABSORBENT ARTICLES

(71) Applicant: Kimberly-Clark Worldwide, Inc., Neenah, WI (US)

(72) Inventors: Vishalkumar Y. Joshi, Sao Paulo (BR); Natalia Hoyos, Medellin (CO); Marcela Rendon, Bogota (CO)

(21) Appl. No.: 16/463,254

(22) PCT Filed: Nov. 30, 2016
(86) PCT No.: PCT/US16/64088

§ 371 Date: May 22, 2019

(51) Int. Cl. A61F 13/84; A61F 13/537; A61L 15/24

(52) U.S. Cl. CPC A61F 13/8405; A61F 13/53743; A61F 2013/530007; A61F 2013/8408; A61L 15/24

(57) ABSTRACT

The present invention relates to disposable absorbent articles that include a bodyside liner, an outer cover and an absorbent core between the bodyside liner and the outer cover. The articles also include a core wrap surrounding the absorbent core. Further, the articles include a shaped form between the core wrap and the absorbent core where the shaped form is a functional structure. The functional structure includes a substrate and a first fixing agent applied to the substrate. The first fixing agent includes an aqueous polymer. The functional structure also includes a functional material that is adjacent to the first fixing agent. A second fixing agent is adjacent to the functional material. The first fixing agent may be a hydrophilic solution including an aqueous polymer selected from polyvinyl alcohol and a cross-linked polyvinyl alcohol. The aqueous polymers may have molecular weights from 1,500,000 to 4,000,000.

### BACKGROUND

A wide variety of limited-use products providing a functional benefit are available in the marketplace. One category of such products are disposable absorbent articles. Disposable absorbent articles are typically a composite of generally two-dimensional materials that are arranged in layers in relation to each other. The two-dimensional materials are nonwoven materials and functional materials. The nonwoven materials are typically polymer-based. The functional materials can include absorbent materials such as “fluff” and superabsorbent and odor-control materials such as charcoal or scented materials.

The functional materials of an absorbent article may be some of the most expensive components of the article and therefore, manufacturers want to maximize the effective use of these components. Further, for absorbent articles, it is desirable to place the functional absorbent materials (superabsorbent and fiber/fluff) in precise locations within the absorbent structure of the article in order to ensure excellent absorbent performance. Significant research has been done by manufacturers of absorbent articles to develop absorbent structures that maximize product performance by minimizing leakage (that is, providing rapid absorption of liquid and having sufficient capacity) while delivering important product attributes such as comfortable fit and keeping wetness away from the skin. When the location and placement of the functional absorbent materials within the absorbent article are well-controlled, product cost is better managed because the amounts of functional absorbent materials are optimized.

There remains a need for a technology to place functional materials in discrete locations and discrete amounts on a substrate for use in products such as disposable absorbent articles. There remains a need to have a higher amount of superabsorbent particles without changing the amount of fluff within the absorbent core of a disposable absorbent article to achieve high absorbency, slim product structure, better dryness and higher storage capacity. Further, there is a need to have zoning of SAP particles within preferred areas of the absorbent core to have higher capacity, fast absorbency and dryness. An obstacle to achieving higher SAP particle amounts is that current absorbent core designs can only hold and distribute a limited amount of fluid. Further, higher amounts of SAP particles can result in pockets of SAP gelling, which have adverse effects on fluid percolation and bulkiness of the absorbent article when wet.

### SUMMARY

The present invention relates to disposable absorbent articles that include shaped forms providing a functional benefit. In one aspect, the present invention relates to a disposable absorbent article that includes a bodyside liner, an outer cover and an absorbent core located between the bodyside liner and the outer cover. The disposable absorbent article also includes a core wrap surrounding the absorbent core. Additionally, the disposable absorbent article includes a shaped form located between the core wrap and the absorbent core. The shaped form is a functional structure that includes a substrate and a first fixing agent applied to the substrate. The first fixing agent includes an aqueous polymer. The functional structure further includes a functional material adjacent the first fixing agent and a second fixing agent adjacent to the functional material. The first fixing agent of the functional structure may be a hydrophilic solution including an aqueous polymer that includes a polyvinyl alcohol or a cross-linked polyvinyl alcohol. The aqueous polymer may have a molecular weight of from 1,500,000 to 4,000,000. The functional material of the functional structure may be one or more of superabsorbent material, pulp fiber material, deodorizing material or fragrance material.

In another aspect, the functional structure has a center and at least one outer edge. The functional material may have a higher concentration at the center of the functional structure than at the outer edge of the functional structure. Put differently, the functional structure may have a concentration gradient of functional material across its surface depending on the intended use of the shaped form/functional material in the disposable absorbent article. In order to form a desired pattern or concentration gradient of functional material, the functional material may be present in a grid-like pattern of square-shaped areas. Discrete placement of the functional material on the substrate may have the benefit of improved performance such as reduced gel-blocking when the functional material is a superabsorbent material.

With regard to the functional structure, the second fixing agent may be a hydrophilic solution of an aqueous polymer selected from polyvinylpyrrolidone in a C1 to C3 alcohol. The aqueous polymer of the second fixing agent may have a molecular weight of at least 30,000.

The shaped form of the functional structure may be a tube-like, cylindrical shape or an accordion-like, pleated shape. The size of the shaped form may be selected based on the desired purpose and performance of the shaped form in the disposable absorbent article. The disposable absorbent article may include a plurality of shaped forms. Each shaped form may have a different size (meaning, the sizes of shaped forms in a disposable absorbent article do not need to be uniform). The shaped form may have a length from 0.3 centimeters to 30 centimeters.

In another aspect, the present invention relates to a disposable absorbent article that includes a bodyside liner, an outer cover and an absorbent core between the bodyside liner and the outer cover. The disposable absorbent article also includes a core wrap surrounding the absorbent core and a surge layer between the bodyside liner and the core wrap. The disposable absorbent article also includes a shaped form between the surge layer and the core wrap. The shaped form is a functional structure that includes a substrate and a first fixing agent applied to the substrate. The first fixing agent of the functional structure includes an aqueous polymer. The functional structure further includes a functional material adjacent the first fixing agent and a second fixing agent adjacent to the functional material. The first fixing agent may be a hydrophilic solution that includes an aqueous polymer including a polyvinyl alcohol or a cross-linked polyvinyl alcohol. The aqueous polymers of the first fixing agent may have molecular weights from 1,500,000 to 4,000,000.

The functional material of the functional structure may be one or more of a superabsorbent material, pulp fiber material, deodorizing material and fragrance material. As with the aspect of the invention described above, the functional structure may have a center and at least one outer edge. Depending on the desired function and performance of the functional structure, the functional material may have a higher concentration at the center of the functional structure than at the outer edge of the functional structure. This essentially creates a concentration gradient of the functional material within the functional structure. In one example, the functional material may be present in a grid-like pattern of square-shaped areas. Discrete placement of the functional material on the substrate may have the benefit of improved performance such as reduced gel-blocking when the functional material is a superabsorbent material.

With regard to the functional structure of this aspect, the second fixing agent may be a hydrophilic solution of an aqueous polymer selected from polyvinylpyrrolidone in a C1 to C3 alcohol. The aqueous polymer of the second fixing agent may have a molecular weight of at least 30,000.

The shaped form of the functional structure may be a tube-like, cylindrical shape or an accordion-like, pleated shape. The size of the shaped form may be selected based on the desired purpose and performance of the shaped form in the disposable absorbent article. The disposable absorbent article may include a plurality of shaped forms. Each shaped form may have a different size (meaning, the sizes of shaped forms in a disposable absorbent article do not need to be uniform). The shaped form may have a length from 0.3 centimeters to 30 centimeters.

Other features and aspects of the present disclosure are discussed in greater detail below.

### BRIEF DESCRIPTION OF THE DRAWINGS

A full and enabling disclosure of the present invention, including the best mode thereof to one skilled in the art, is set forth more particularly in the remainder of the specification, including reference to the accompanying figures, in which:

FIG. 1 is a cross-sectional view of a functional structure of the invention;

FIG. 2 is a plan view of a substrate with a template laid over the substrate;

FIGS. 3A and 3B are plan views of a substrate with a functional material fixed to it; FIGS. 3A and 3B illustrate examples of different concentration gradients of the functional material on the substrate.

FIG. 4 is a perspective view of an exemplary absorbent article to show the general construction.

FIG. 5 is a cross-sectional view of the exemplary absorbent article of FIG. 4.

FIG. 6 is a plan view of an embodiment of a functional structure in accordance with the present disclosure.

FIG. 7 is a plan view of a tube-like (cylindrical) shaped forms that may be used in a disposable absorbent article.

FIG. 8 is a plan view of tube-like (cylindrical) shaped forms placed between representative component layers of a disposable absorbent article.

Repeat use of reference characters in the present specification and drawings is intended to represent the same or analogous features or elements of the present invention.

### Definitions

The term “absorbent article” refers herein to an article which may be placed against or in proximity to the body (i.e., contiguous with the body) of the wearer to absorb and contain various liquid, solid, and semi-solid exudates discharged from the body. Such absorbent articles, as described herein, are intended to be discarded after a limited period of use instead of being laundered or otherwise restored for reuse. It is to be understood that the present disclosure is applicable to various disposable absorbent articles, including, but not limited to, diapers, training pants, youth pants, swim pants, feminine hygiene products, including, but not limited to, menstrual pads, incontinence products, medical garments, surgical pads and bandages, other personal care or health care garments, and the like without departing from the scope of the present disclosure.

The term “surge layer” refers herein to a layer capable of accepting and temporarily holding liquid body exudates to decelerate and diffuse a surge or gush of the liquid body exudates and to subsequently release the liquid body exudates therefrom into another layer or layers of the absorbent article.

The term “bonded” refers herein to the joining, adhering, connecting, attaching, or the like, of two elements. Two elements will be considered bonded together when they are joined, adhered, connected, attached, or the like, directly to one another or indirectly to one another, such as when each is directly bonded to intermediate elements.

The term “carded web” refers herein to a web containing natural or synthetic staple fibers typically having fiber lengths less than about 100 mm. Bales of staple fibers can undergo an opening process to separate the fibers which are then sent to a carding process which separates and combs the fibers to align them in the machine direction after which the fibers are deposited onto a moving wire for further processing. Such webs are usually subjected to some type of bonding process such as thermal bonding using heat and/or pressure. In addition to or in lieu thereof, the fibers may be subject to adhesive processes to bind the fibers together such as by the use of powder adhesives. The carded web may be subjected to fluid entangling, such as hydroentangling, to further intertwine the fibers and thereby improve the integrity of the carded web. Carded webs, due to the fiber alignment in the machine direction, once bonded, will typically have more machine direction strength than cross machine direction strength.

The term “film” refers herein to a thermoplastic film made using an extrusion and/or forming process, such as a cast film or blown film extrusion process. The term includes apertured films, slit films, and other porous films which constitute liquid transfer films, as well as films which do not transfer fluids, such as, but not limited to, barrier films, filled films, breathable films, and oriented films.

The term “g/cc” refers herein to grams per cubic centimeter.

The term “gsm” refers herein to grams per square meter.

The term “hydrophilic” refers herein to fibers or the surfaces of fibers which are wetted by aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by Cahn SFA-222 Surface Force Analyzer System, or a substantially equivalent system. When measured with this system, fibers having contact angles less than 90 degrees are designated “wettable” or hydrophilic, and fibers having contact angles greater than 90 degrees are designated “nonwettable” or hydrophobic.

The term “liquid impermeable” refers herein to a layer or multi-layer laminate in which liquid body exudates, such as urine, will not pass through the layer or laminate, under ordinary use conditions, in a direction generally perpendicular to the plane of the layer or laminate at the point of liquid contact.

The term “liquid permeable” refers herein to any material that is not liquid impermeable.

The term “meltblown” refers herein to fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter, which can be a microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al., which is incorporated herein by reference. Meltblown fibers are microfibers which may be continuous or discontinuous, are generally smaller than about 0.6 denier, and may be tacky and self-bonding when deposited onto a collecting surface.

The term “nonwoven” refers herein to materials and webs of material which are formed without the aid of a textile weaving or knitting process. The materials and webs of materials can have a structure of individual fibers, filaments, or threads (collectively referred to as “fibers”) which can be interlaid, but not in an identifiable manner as in a knitted fabric. Nonwoven materials or webs can be formed from many processes such as, but not limited to, meltblowing processes, spunbonding processes, carded web processes, etc.

The term “pliable” refers herein to materials which are compliant and which will readily conform to the general shape and contours of the wearer's body.

The term “spunbond” refers herein to small diameter fibers which are formed by extruding molten thermoplastic material as filaments from a plurality of fine capillaries of a spinnerette having a circular or other configuration, with the diameter of the extruded filaments then being rapidly reduced by a conventional process such as, for example, eductive drawing, and processes that described in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Peterson, and U.S. Pat. No. 3,542,615 to Dobo et al., each of which is incorporated herein in its entirety by reference. Spunbond fibers are generally continuous and often have average deniers larger than about 0.3, and in an embodiment, between about 0.6, 5 and 10 and about 15, 20 and 40. Spunbond fibers are generally not tacky when they are deposited on a collecting surface.

The term “superabsorbent” refers herein to a water-swellable, water-insoluble organic or inorganic material capable, under the most favorable conditions, of absorbing at least about 15 times its weight and, in an embodiment, at least about 30 times its weight, in an aqueous solution containing 0.9 weight percent sodium chloride. The superabsorbent materials can be natural, synthetic and modified natural polymers and materials. In addition, the superabsorbent materials can be inorganic materials, such as silica gels, or organic compounds, such as cross-linked polymers.

The term “thermoplastic” refers herein to a polymeric material which becomes pliable or moldable above a specific temperature and returns to a solid state upon cooling.

### DETAILED DESCRIPTION

It is to be understood by one of ordinary skill in the art that the present discussion is a description of exemplary embodiments only, and is not intended as limiting the broader aspects of the present disclosure.

In general, the present disclosure is directed to functional structures that are used within absorbent articles. The functional structures of the invention may be used in one or more locations within the absorbent article. For purposes of organizing the description and explanation of the invention, the functional structures will be described followed by a description of the absorbent articles in which the functional structures may be used. FIG. 1 provides a representative illustration of a functional structure 100 of the invention. The functional structure 100 is constructed of primarily two-dimensional layers; the manner in which the relative thicknesses of the layers are portrayed in FIG. 1 is representative only and the invention is not intended to be limited by particular layer thicknesses. The base of the functional structure 100 is a substrate 200; the substrate 200 is the layer upon which the other layers are formed. The substrate 200 may be formed of a suitable nonwoven material such as a carded web material, a spunbond material, a meltblown material, a tissue material, a coform material, a spunlace material or a combination of one or more of such materials. The size and shape of the substrate 200 is selected based on the size of the absorbent article in which the functional structure 100 is to be used and the intended location of the functional structure 100 within the absorbent article. The shape of the substrate 200 can also be selected based on optimizing the intended purpose of the functional structure 100. The shape of the substrate 200 may be polygonal such as rectangular or triangular or the shape may be a closed shape that includes curves such as a circle. Further, the shape of the substrate 200 may include straight line edges combined with curved edges depending on the intended purpose of the functional structure 100.

The next layer of the functional structure is a first fixing agent 300. The purpose of the first fixing agent 300 is to prepare the substrate for application of the functional material 400. The first fixing agent 300 may be selected from solutions of aqueous polymers, including polyvinyl alcohols and cross-linked polyvinyl alcohols having molecular weights between 1,500,000 and 4,000,000. The cross-linked polyvinyl alcohols may be selected from polyvinyl alcohols mixed with aqueous solution of Borax (sodium borate, sodium tetraborate, or disodium tetraborate). In an exemplary embodiment, the first fixing agent 300 is a 10% aqueous solution of polyvinyl alcohol having a molecular weight of about 2,000,000. The first fixing agent 300 may form a continuous layer over the substrate 200, or the first fixing agent 300 may be in discontinuous locations on the substrate 200. The first fixing agent 300 may be applied in a pattern on the substrate 200.

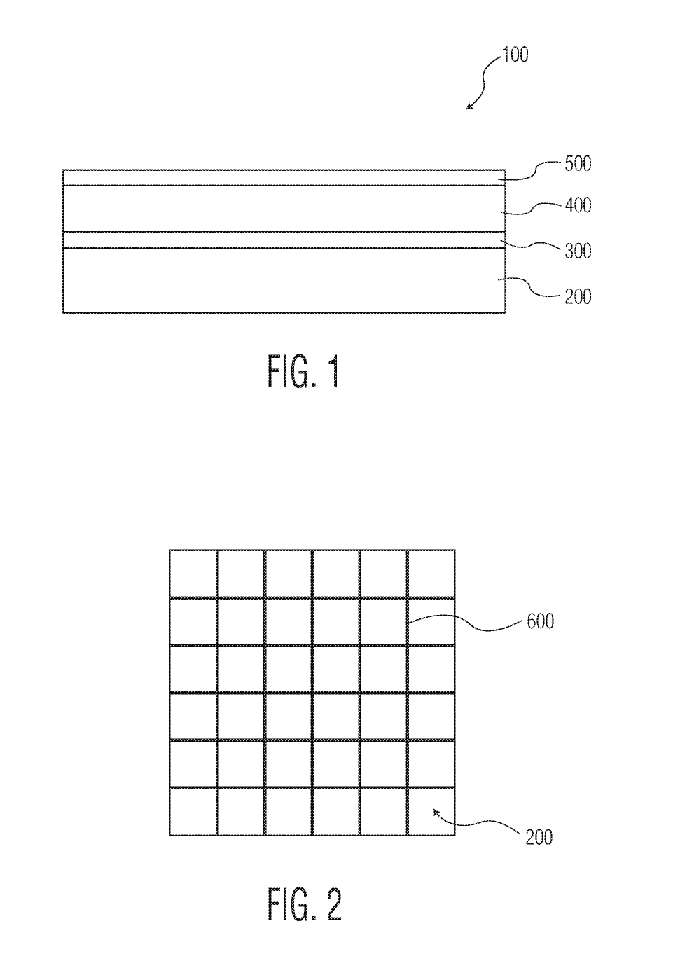

The next layer of the functional structure 100 adjacent to the first fixing agent 300 is the functional material 400. The functional material 400 is the component of the functional structure 100 that provides the “working” or “active” purpose of the functional structure 100. The functional material 400 may be selected from absorbent materials such as superabsorbent material and cellulose fiber, also referred to as pulp. The functional material 400 may also be selected from a deodorizing material, such as charcoal, or a fragrance material. The functional material 400 may form a continuous layer over the first fixing agent 300, or the functional material 400 may be in discontinuous locations on the first fixing gent 300. Further, as shown in FIG. 2, the functional material 400 may be in a pattern on the first fixing agent 300 and the substrate 200. FIG. 2 shows a substrate 200 with a grid-like template 600 placed on top of it prior to formation of the first fixing agent 300 layer and the functional material 400 layers. While the template 600 shown in FIG. 2 has straight lines that are spaced equidistantly from each other to form a grid of same-sized squares, for functional structures 100 of the invention may utilize templates 600 of whatever shapes and patterns are appropriate for the intended purpose of the functional structure 100. A purpose of using a template 600 in the formation of a functional structure 100 of the invention is to place the functional material 400 is precisely defined locations on the substrate 200. The template 600 may also be used to place the functional material 400 in different concentrations on the substrate 200. For example, a template 600 may be used to form a concentration gradient of the functional material 400 from the center of the functional structure 100 (having the higher concentration of functional material 400) to the outer edges of the functional structure 100 (having gradually decreasing concentrations of functional material 400 moving outward from the center of the functional structure 100). Generally speaking, the template 600 is used to place the functional material 400 in the desired locations and in the desired concentrations on the substrate 200 to maximize the performance of the functional structure 100. The first fixing agent 300 may be applied to the substrate 200 in the pattern provided by a template 600 prior to placement of the functional material 400.

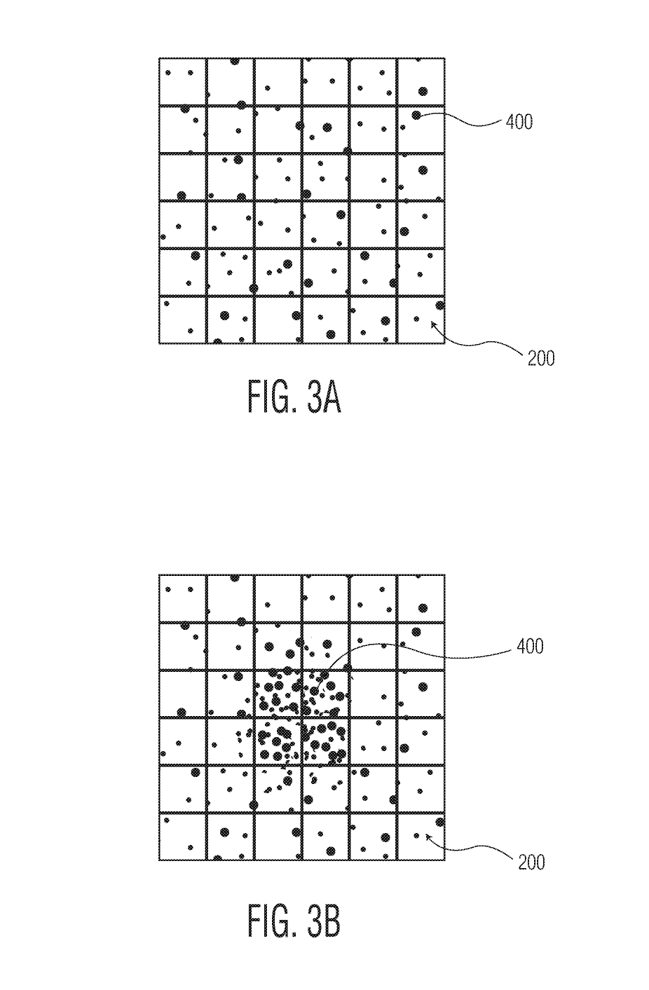

FIG. 3 depicts exemplary embodiments of the present invention in which a template 600 is used to place both the first fixing agent 300 and the functional material 400 on the substrate 200. In FIG. 3A, the formed pattern is a grid of squares, each having equal concentrations (or amounts) of the functional material 400. The functional material 400 shown in FIG. 3A and FIG. 3B is a superabsorbent material. In FIG. 3B, the formed pattern is also a grid of squares, but there is a concentration gradient of the functional material 400. The exemplary gradient of functional material 400 has a higher concentration of functional material 400 at the center of the substrate 200 and a gradually decreasing concentration of the functional material 400 moving away from the center of the substrate 200. The gradient of functional material 400 could also be reversed to have the higher concentration (corresponding to a higher quantity) of functional material 400 on the outward edges of the substrate 200 with a gradually decreasing concentration of the functional material 400 moving toward the center of the substrate 200. Desired patterns, placements and concentrations of functional material 400 on the substrate 200 may be selected based on the intended purpose and performance of the functional structure 100.

After the layer of the functional material 400, the functional structures 100 of the invention include a second fixing agent 500. The second fixing agent 500 secures or seals the functional material 400 in its location on the substrate 200. The second fixing agent 500 may be applied in a continuous or discontinuous layer on the functional material 400. The second fixing agent 500 may be selected from alcoholic or aqueous solutions of polymers. In an exemplary embodiment of the present invention, the second fixing agent 500 is selected from a polyvinylpyrrolidone that is soluble in methanol, ethanol or propanol and has a molecular weight of at least 30,000. In a more specific exemplary embodiment, the second fixing agent 500 is a 10% solution of polyvinylpyrrolidone in ethanol and having a molecular weight of 90,000.

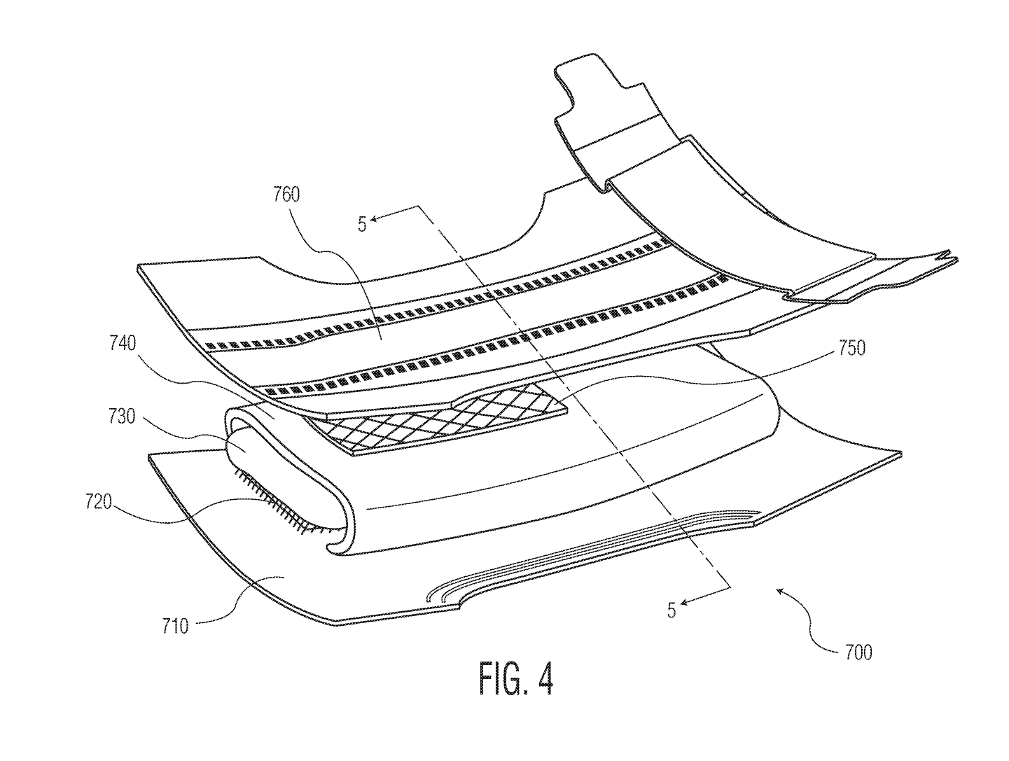

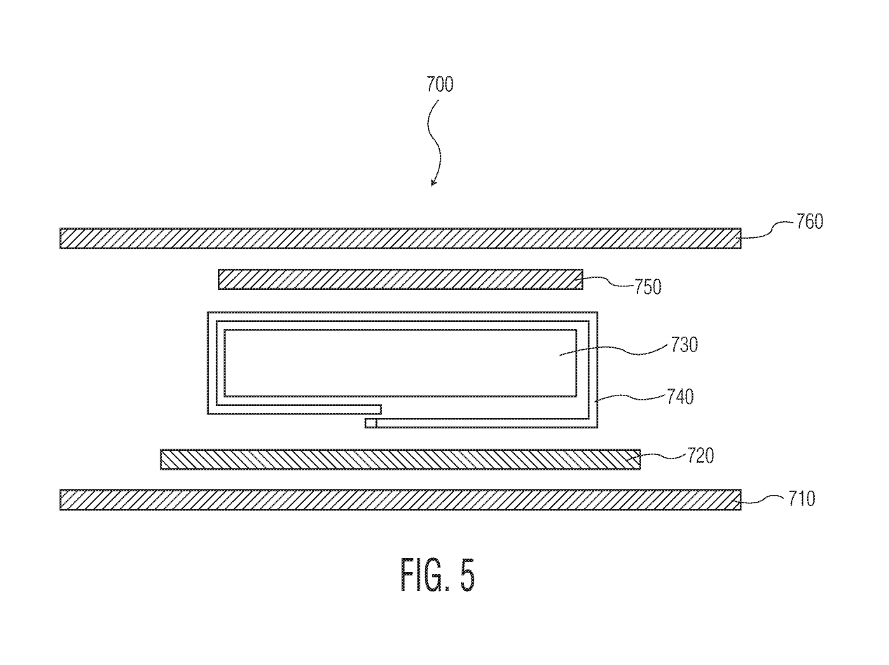

As described herein, the functional structures 100 of the invention may be used as one or more components of a disposable absorbent article. A simplified version (that is, a version not showing every feature of a modern disposable absorbent article) of a disposable diaper type of absorbent article is shown in FIGS. 4 and 5. The purpose of the simplified version is to provide context for where a functional structure 100 of the invention might be used as a component. Referring to FIG. 4, a disposable absorbent article 700 generally includes an outer cover 710 that includes an exterior surface and an interior surface. Located adjacent the interior surface is an absorbent core 730. The disposable absorbent article 700 may also include a liquid permeable bodyside liner 760. An absorbent core 730 may be placed in between the outer cover 710 and the bodyside liner 760.

The disposable absorbent article 700 as shown in FIG. 4 may be made from various materials. The outer cover 710 may be made from a material that is substantially liquid impermeable, and can be elastic, stretchable or nonstretchable. The outer cover 710 may be a single layer of liquid impermeable material, or may include a multi-layered laminate structure in which at least one of the layers is liquid impermeable. For instance, the outer cover 710 may include a liquid permeable outer layer and a liquid impermeable inner layer that are suitably joined together by a laminate adhesive. For example, in one embodiment, the liquid permeable outer layer may be a spunbond polypropylene nonwoven web. The spunbond web may have, for instance, a basis weight of from about 15 gsm to about 25 gsm. The inner layer, on the other hand, may be both liquid and vapor impermeable, or can be liquid impermeable and vapor permeable. The inner layer is suitably manufactured from a thin plastic film, although other flexible liquid impermeable materials may also be used. The inner layer prevents waste material from wetting articles such as bedsheets and clothing, as well as the wearer and caregiver. A suitable liquid impermeable film may be a polyethylene film having a thickness of about 0.2 mm. A suitable breathable material that may be used as the inner layer is a microporous polymer film or a nonwoven fabric that has been coated or otherwise treated to impart a desired level of liquid impermeability. Other “non-breathable” elastic films that may be used as the inner layer include films made from block copolymers, such as styrene-ethylene-butylene-styrene or styrene-isoprene-styrene block copolymers.

As described above, the absorbent core 730 is positioned in between the outer cover 710 and the bodyside liner 760. The bodyside liner 760 is suitably compliant, soft feeling, and non-irritating to the wearer's skin. The bodyside liner 760 may be manufactured from a wide variety of web materials, such as synthetic fibers, natural fibers, a combination of natural and synthetic fibers, porous foams, reticulated foams, apertured plastic films, or the like. Various woven and nonwoven fabrics can be used for the bodyside liner 760. For example, the bodyside liner 760 may be made from a meltblown or spunbonded web of polyolefin fibers. The bodyside liner 760 may also be a bonded-carded web composed of natural and/or synthetic fibers. A suitable liquid permeable bodyside liner 760 is a nonwoven bicomponent web having a basis weight of about 27 gsm. The nonwoven bicomponent can be a spunbond bicomponent web, or a bonded carded bicomponent web. Suitable bicomponent staple fibers include a polyethylene/polypropylene bicomponent fiber. In this particular embodiment, the polypropylene forms the core and the polyethylene forms the sheath of the fiber. Other fiber orientations, however, are possible.

The material used to form the absorbent core 730, for example, may include cellulosic fibers (e.g., wood pulp fibers), other natural fibers, synthetic fibers, woven or nonwoven sheets, scrim netting or other stabilizing structures, superabsorbent material, binder materials, surfactants, selected hydrophobic materials, pigments, lotions, odor control agents or the like, as well as combinations thereof. In a particular embodiment, the absorbent core 730 is a matrix of cellulosic fluff and superabsorbent hydrogel-forming particles. The cellulosic fluff may comprise a blend of wood pulp fluff. One preferred type of fluff is identified with the trade designation CR 1654, available from US Alliance Pulp Mills of Coosa, Ala., USA, and is a bleached, highly absorbent wood pulp containing primarily soft wood fibers. As a general rule, the superabsorbent material is present in the absorbent core 730 in an amount of from about 0 to about 90 weight percent based on total weight of the web. The superabsorbent material may even comprise 100 weight percent of the absorbent core 730. The web may have a density within the range of about 0.1 to about 0.45 grams per cubic centimeter.

Superabsorbent materials are well known in the art and may be selected from natural, synthetic, and modified natural polymers and materials. The superabsorbent materials may be inorganic materials, such as silica gels, or organic compounds, such as crosslinked polymers. Typically, a superabsorbent material is capable of absorbing at least about 15 times its weight in liquid, and suitably is capable of absorbing more than about 25 times its weight in liquid. Suitable superabsorbent materials are readily available or have previously been available from various suppliers. For example, FAVOR SXM 880 superabsorbent is available (or was previously available) from Stockhausen, Inc., of Greensboro, N.C., USA; and Drytech 2035 is available (or was previously available) from Dow Chemical Company, of Midland, Mich., USA.

In addition to cellulosic fibers and superabsorbent materials, the absorbent core 730 may also contain adhesive elements and/or synthetic fibers that provide stabilization and attachment when appropriately activated. Additives such as adhesives may be of the same or different aspect from the cellulosic fibers; for example, such additives may be fibrous, particulate, or in liquid form; adhesives may possess either a curable or a heat-set property. Such additives may enhance the integrity of the bulk absorbent structure, and alternatively or additionally may provide adherence between facing layers of the folded structure.

The absorbent materials may be formed into a web structure by employing various conventional methods and techniques. For example, the absorbent core 730 may be formed with a dry-forming technique, an airlaying technique, a carding technique, a meltblown or spunbond technique, a wet-forming technique, a foam-forming technique, or the like, as well as combinations thereof. Layered and/or laminated structures may also be suitable. Methods and apparatus for carrying out such techniques are well known in the art.

The absorbent core 730 may also be a coform material. The term “coform material” generally refers to composite materials comprising a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff and also superabsorbent particles or fibers, inorganic absorbent materials, treated polymeric staple fibers and the like. Any of a variety of synthetic polymers may be utilized as the melt-spun component of the coform material. For instance, in some embodiments, thermoplastic polymers can be utilized. Some examples of suitable thermoplastics that can be utilized include polyolefins, such as polyethylene, polypropylene, polybutylene and the like; polyamides; and polyesters. In one embodiment, the thermoplastic polymer is polypropylene. Some examples of such coform materials are disclosed in U.S. Pat. No. 4,100,324 to Anderson, et al.; U.S. Pat. No. 5,284,703 to Everhart, et al.; and U.S. Pat. No. 5,350,624 to Georger, et al.; which are incorporated herein in their entirety by reference for all purposes.

In addition to the basic components of an outer cover 710, an absorbent core 730 and a bodyside liner 760 as shown in FIG. 4, disposable absorbent articles 700 may have additional components that improve product performance. For example, disposable absorbent articles often include a core wrap 740 that surrounds (either partially or entirely) the absorbent core 730. The core wrap 740 component typically assists with maintaining the structural integrity of the absorbent core 730 during use of the disposable absorbent article 700. As the absorbent core 730 of the disposable absorbent article 700 absorbs fluid, it may lose shape and integrity; it is also desirable to keep the components of the absorbent core 730 from exiting the absorbent article 700. The core wrap 740 assists with containment and integrity of the absorbent core 730 components. The core wrap 740 is shown in FIG. 4 and its relationship to the absorbent core 730 is visible in the cross-section view of FIG. 5. The core wrap 740 may be bonded to the absorbent core 730. Bonding of the core wrap 740 to the absorbent core 730 may occur via any means known to one of ordinary skill, such as, but not limited to, adhesives. The core wrap 740 may be composed of separate sheets of material which can be utilized to partially or fully encompass the absorbent core 730 and which can be sealed together using a sealing means such as an ultrasonic bonder or other thermochemical bonding means or the use of an adhesive. The core wrap 740 may include, but is not limited to, natural and synthetic fibers such as polyester, polypropylene, acetate, nylon, polymeric materials, cellulosic materials such as wood pulp, cotton, rayon, viscose, LYOCELL® such as from Lenzing Company of Austria, or mixtures of these or other cellulosic fibers, and combinations thereof. Natural fibers may include wool, cotton, flax, hemp, and wood pulp. The material forming the core wrap 740 may be selected from meltblown-spunbond-meltblown fabric, spunbond fabric, meltblown fabric, coform fabric, carded web, bonded-carded web, bicomponent spunbond fabric, spunlace, tissue, and combinations thereof. Further, the core wrap 740 may be made of a spunbond-meltblown-spunbond (“SMS”) material, such as a 10 gsm spunbond-meltblown-spunbond material.

The core wrap 740 may be less hydrophilic than the absorbent core 730, but sufficiently porous to permit liquid body exudates to penetrate through the core wrap 740 to reach the absorbent core 730. Desirably, the core wrap 740 has sufficient structural integrity to withstand its own wetting and the wetting of the absorbent core 730. In order to support this functional property of the core wrap 740, a wet strength agent may be applied to the core wrap 740. A non-limiting example of a wet strength agent may be Kymene 6500 (557LK) or equivalent available from Ashland Inc. of Ashland, Ky., U.S.A. Similarly, a surfactant may be included in the core wrap 740.

Another beneficial component that is typically included in the structure of a disposable absorbent article 700 is a spacer layer 720. The spacer layer 720 is typically located between the absorbent core 730 and the outer cover 710. The purpose of the spacer layer 720 is to prevent moisture from migrating from the absorbent core 730 outward toward and through the outer cover 710. When this type of moisture migration occurs, the outer cover 710 may feel damp, which can be perceived as product leakage. The dimensions of the spacer layer 720 typically match the dimensions of the absorbent core 730 and are typically smaller than the dimensions of the outer cover 710. Disposable absorbent articles are often constructed to have breathable outer covers 710 that allow for the passage of water vapor (but not liquids); the spacer layer 720 is typically sized to not interfere with this function of the outer cover 710. The spacer layer may be constructed of known nonwoven materials, including spunbond-meltblown-spunbond materials.

Conventional disposable absorbent articles 700 typically include an additional component known as the surge layer 750 which, like the core wrap 740 and the spacer layer 720, assists with fluid management within the absorbent article 700. Typically located between the bodyside liner 760 and the absorbent core 730 (or between the bodyside liner 760 and the core wrap 740, if a core wrap 740 is present), the surge layer 750 helps decelerate and diffuse surges or gushes of liquid body exudates penetrating the bodyside liner 760. The surge layer 750 accomplishes this purpose by taking in and distributing body exudates for absorption by the absorbent core 730. As shown in FIG. 4, the surge layer 750 is positioned underneath the location of the bodyside liner 760 where fluid surges occur for both males and females. As shown in the cross-sectional view of FIG. 5, the approximate dimensions of the surge layer 750 are smaller than the dimensions of the bodyside liner 760 and approximately the same as the width of the absorbent core 730. The surge layer 750 is typically shorter in length than the absorbent core 730. The surge layer 750 may be in contact with and/or bonded with the bodyside liner 760. Bonding of the surge layer 750 to the bodyside liner 760 may occur through the use of an adhesive and/or point fusion bonding. The point fusion bonding can be selected from ultrasonic bonding, pressure bonding, thermal bonding, and combinations thereof. The point fusion bonding can be provided in any pattern as deemed suitable.

The surge layer 750 may include natural fibers, synthetic fibers, superabsorbent material, woven material, nonwoven material, wet-laid fibrous webs, a substantially unbounded airlaid fibrous web, an operatively bonded, stabilized-airlaid fibrous web, or the like, as well as combinations thereof. In an embodiment, the surge layer 750 may be formed from a material that is substantially hydrophobic, such as a nonwoven web composed of polypropylene, polyethylene, polyester, and the like, and combinations thereof. The surge layer 750 may be formed of one or more materials selected from meltblown-spunbond-meltblown fabric, spunbond fabric, meltblown fabric, coform fabric, carded web, bonded-carded web, bicomponent spunbond fabric, spunlace, tissue, and combinations thereof.

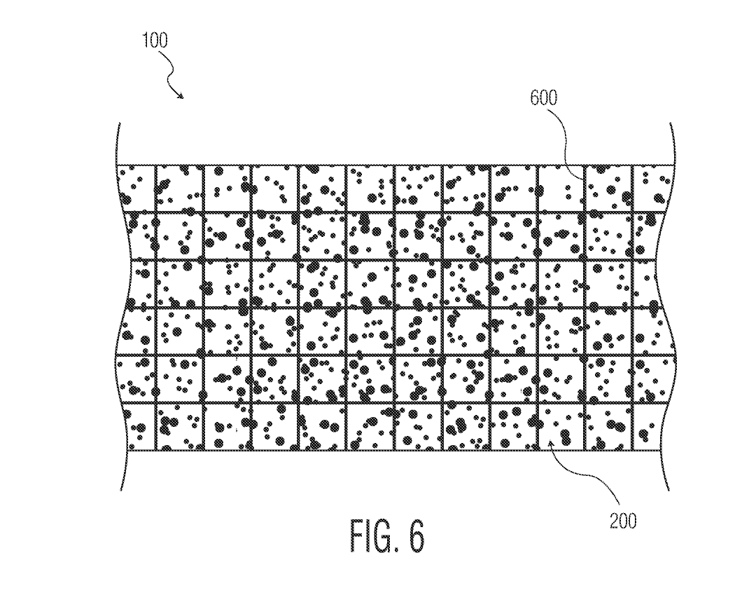

The foregoing description of a disposable absorbent article 700 provides the context for how the functional structures 100 of the invention may be used. The functional structures 100 of the invention may be used to construct or to be a component of any one of the outer cover 710, the spacer layer 720, the absorbent core 730, the core wrap 740, the surge layer 750 and/or the bodyside liner 760. The functional structure 100 may be used to form all or part of one or more of these disposable absorbent article 700 components depending on desired placement of a functional material 400. Consistent with the functional purpose already fulfilled, two components that would be expected to include executions of the functional structures 100 are the core wrap 740 and the absorbent core 730. Without limiting the intended breadth of the invention, these two components would be expected to demonstrate the benefits associated with the present invention because of their existing purpose of absorbing fluids. Particularly if the functional material 400 is an absorbent material such as superabsorbent material or pulp fibers, the core wrap 740 and absorbent core 730 could have improved efficiency by including one or more functional structures 100. In the context of a core wrap 740 or absorbent core 730, the functional structure 100 provides the benefit of better controlling the placement of absorbent materials to prevent such efficiency-limiting phenomena as gel-blocking. Gel-blocking is a phenomenon that occurs when the swelling of a superabsorbent polymer during the absorption of fluid blocks the passage of fluid into the center or along the length of the absorbent core 730, thereby reducing the absorption capacity. By using one or more of the functional structures 100 of the invention as the core wrap 740 or as the absorbent core 730, the negative effects of gel-blocking may be reduced and the absorption capacity of the disposable absorbent article 700 improved. FIG. 6 representatively illustrates use of a functional structure 100 as the absorbent core 730 for a disposable absorbent article 700. The functional structure 100 of FIG. 6 was formed using a grid-shaped template 600 that directed placement of the functional material 400 (superabsorbent particles) into discrete, square-shaped areas on the substrate 200. The resulting functional structure 100 has a more controlled distribution and placement of the superabsorbent functional material 400. When used as an absorbent core 730, this functional structure 100 will make more efficient use of the functional material 400 and will have greater absorption capacity because gel-blocking will be reduced. In use, disposable absorbent articles 700 including a functional structure 100 can be expected to perform better by having fewer leaks, less flowback of fluid from the absorbent core 730 back through the bodyside liner 760 toward the skin of the wearer and increased capacity.

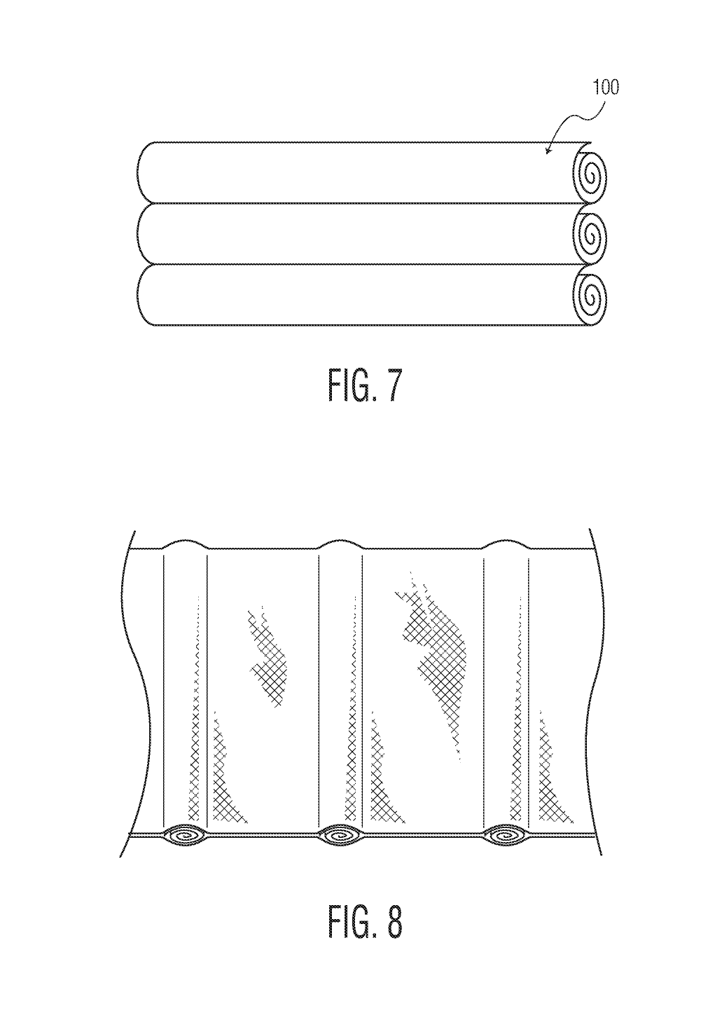

Another aspect of the present invention relates to forming three-dimensional shaped forms using the functional structures 100 of the invention. The functional structures 100 of the invention are flexible and moldable into other forms that may be placed within the structure of a disposable absorbent article 700. For example, the functional structures 100—which are primarily two-dimensional—may be rolled upon themselves to create tube-like (or “cylindrical”) shaped forms. FIG. 7 representatively shows three functional structures 100 that have been rolled upon themselves to form tube-like (cylindrical) shaped forms. Alternatively, the functional structures may be pleated upon themselves to create accordion-like shaped forms. Once the shaped forms are created, one or more of the shaped forms may be placed in different locations within a disposable absorbent article 700. FIG. 8 representatively shows how three of the shaped forms may be placed between representative layers of a disposable absorbent article 700. For example, the arrangement shown in FIG. 8 could be the shaped forms (functional structures 100) placed between two surge layers 750. Alternatively, the arrangement of FIG. 8 could be placement of the shaped forms between a surge layer 750 and a core wrap 740. In another embodiment, the shaped forms may be placed between a core wrap 740 and the absorbent core 730. It is also possible to place the shaped forms between a bodyside liner 760 and a surge layer 750. Depending on the intended purpose of the functional material 400 in the functional structures 100 making up the shaped forms, the shaped forms may also be placed between a core wrap 740 and a spacer layer 720 or between a spacer layer 720 and an outer cover 710. This arrangement may be desirable if the functional material 400 is an deodorizing/odor control or fragrance material.

The shaped forms may be placed in various patterns and configurations between the various layers of a disposable absorbent article 700 (the various layers between which the shaped forms may be placed being described in the previous paragraph). In one aspect, the shaped forms may be placed in parallel with each other with space between each shaped form, as shown in FIG. 8. With the configuration of shaped forms shown in FIG. 8, the placement of the shaped forms creates channels for directing the body exudates being absorbed by the disposable absorbent article 700. The shaped forms may also be cut into smaller length pieces for more discrete placement in different locations. The length of a shaped form may be from 0.3 centimeters to 30 centimeters and may be selected based on intended purpose and the dimensions of the disposable absorbent article in which the one or more shaped forms are used. The shaped forms may be placed in any number of configurations between the various layers of a disposable absorbent article 700. Selection of the configuration of shaped forms may be made depending on the intended purpose of the functional structures 100 and the placement of shaped forms that best enables maximum efficacy of the functional material 400.

Various aspects of the present disclosure may be better understood with reference to the following examples.

### Example 1

Preparation of first fixing agent 300 and a second fixing agent 500 for use in a functional structure 100 of the invention. Preparation of 10% aqueous polyvinyl alcohol (“PVA”; first fixing agent 300), 10% polyvinylpyrrolidone (“PVP”) ethanol solution and cross-linked PVA gel. To prepare a 10% aqueous solution of PVA, 10 grams of PVA granules are placed in a beaker. Take 1000 ml water and raise temperature to 100° C. over a heating mantle. Next, 1000 milliliters of 100° C. water are added slowly to the PVA granules while stirring to ensure complete dissolution. The solution is then cooled and may be stored at room temperature until use. To prepare a 10% PVP solution (second fixing agent 500), 1000 milliliters of ethanol are added to 10 grams of PVP and stirred at room temperature for 1 hour. After complete dissolution of PVP, the solution may be stored at room temperature until use. To prepare a cross-linked PVA gel (first fixing agent 300), add 1% borax aqueous solution to 10% aqueous solution of PVA and stir at room temperature for 10 minutes. Add sufficient water to dilute the formed cross-linked gel. The cross-linked PVA gel solution may be stored at room temperature until use.

### Example 2

Preparation of a primarily two-dimensional (“2D”) functional structure 100 that may be used as a spunbond or SMS core wrap 740 that contains superabsorbent (“SAP”) particles. A layer-by-layer (“L-B-L”) composite formation using a soft template method is used to form the functional core wrap 740 (see also Example 10 below). First, an aqueous PVA or cross-linked PVA gel layer is applied on a 50 centimeter by 20 centimeter (“cm”) piece of spunbond material (basis weight, 12 gsm) or SMS (also 12 gsm) (spunbond and SMS material are the substrate 200). The concentration of PVA on the spunbond or SMS material may be selected and adjusted based on final SAP particle concentration. A concentration of between 0.1 gsm to 1.5 gsm (dry weight) may be achieved. Next, the desired amount of SAP particles (0.1 grams to 6 grams; SAP particles are the functional material 400) were sprinkled over the spunbond/SMS material. The SAP particles immediately stick to the spunbond layer because of the PVA/cross-linked PVA binder layer (acting as the first fixing agent 300). The aqueous PVA solution or cross-linked gel solution acts as a binder and controls swelling of the SAP particles. After application of the SAP particles, deposition swelling was very minimal and difficult to observe with naked eye. Next, a 10% ethanol-PVP solution (the second fixing agent 500) was sprayed over the SAP particle layer using a spray paint gun. The pressure ranged between 1 millibar to 3.5 millibar. The concentration of PVP over the SAP particles may be selected and adjusted based on the final SAP particle concentration. During experiments, a concentration of between 0.1 gsm to 1.5 gsm (dry weight) may be achieved. The prepared L-B-L functional structure dried at room temperature around 30 min and was usable to be a component in a disposable absorbent article. The final SAP particle concentration over the spunbond/SMS material may be between 0.1 gsm and 175 gsm.

### Example 3

Preparation of functional structure 100 that may be used as a tissue, coform or spunlace core wrap 740 that contains superabsorbent (“SAP”) particles. Same as Example 2, except a tissue material (25 gsm) is used to form the substrate 200 of the functional structure 100 to be used as a core wrap 740.

### Example 4

Preparation of three-dimensional (“3D”), shaped form of a functional structure 100 in the shape of a tube (that is, a cylindrical shape) that may be used between a surge layer 750 and a spunbond or SMS core wrap 740 that contains superabsorbent (“SAP”) particles and fluff fibers. The functional structures 100 of either Example 2 or Example 3 can be rolled to form tubes having an internal diameter of between 3 millimeters and 3 centimeters and a length of between 1 centimeter and 30 centimeters. The tube-like shaped forms (functional structures 100) may be placed between a surge layer 750 and a core wrap 740 such that the length of the tube-like shaped form (functional structure 100) is parallel with the length of the absorbent core 730. The length of the absorbent core 730 is typically the longer of the two dimensions.

### Example 5

The tube-like shaped form (functional structure 100) prepared in Example 4 may be cut into smaller length pieces and may be used to construct different patterns on top of the core wrap 740. These pieces can be from 3 millimeters to 10 millimeters and placed with their length parallel or perpendicular to the length of the absorbent core.

### Example 6

Preparation of three-dimensional (“3D”), shaped form of a functional structure 100 that may be used as a spunbond or SMS core wrap 740 that contains superabsorbent (“SAP”) particles. A layer-by-layer (“L-B-L”) composite formation using a soft template method is used to form the functional core wrap 740 (see also Example 11 below). First, place the desired three-dimensional silicone sheet (as the template/mold 600, having desired shapes and geometries) over a 50 cm by 20 cm piece of spunbond (12 gsm) or SMS (12 gsm) material. Next, spray the aqueous PVA solution or cross-linked PVA gel solution over the 3D silicone template/mold as explained in example 11. The concentration of PVA solution (the first fixing agent 300) over the spunbond or SMS material may be selected and adjusted based on the final SAP particle concentration. A concentration of between 0.1 gsm to 1.5 gsm (dry weight) for the first fixing agent 300 may be achieved. Next, the desired amount of SAP particles (0.1 grams to 6 grams; the functional material 400) may be sprinkled over the 3D silicone template mold 600. A zoning or density gradient of the SAP particles may be formed by sprinkling more or fewer particles in particular sections or areas of the template 600. The SAP particles (the functional material 400) will immediately stick to the spunbond layer (the substrate 200) by the first fixing agent 300 of PVA solution or cross-linked PVA binder solution. Aqueous PVA solution or cross-linked PVA gel solution may act as binder and may control swelling of the SAP particles. After application of the SAP particles, deposition swelling may be very minimal and difficult to observe with naked eye. Next, a 10% ethanol-PVP solution (the second fixing agent 500) is sprayed over the 3D silicone template/mold 600 and the SAP particles layer using a spray paint gun. The pressure may range from between 1 millibar to 3.5 millibar. The concentration of PVP solution over the SAP particles may be selected and adjusted based on the final concentration of SAP particles. A concentration of between 0.1 gsm to 1.5 gsm (dry weight) of the PVP solution may be achieved. The 3D functional structure 100 containing SAP particles usable as a core wrap 740 in a disposable absorbent article 700 may be dried at room temperature around 30 min. The final concentration of SAP particles over the spunbond or SMS material may be between 0.1 gsm and 175 gsm.

### Example 7

Preparation of 3D, shaped form of a functional structure 100 that may be used as a tissue, coform or spunlace core wrap 740 that contains superabsorbent (“SAP”) particles. Same as Example 6, except a tissue material (25 gsm) is used to form the substrate 200 of the functional structure 100 to be used as a core wrap 740.

### Example 8

Preparation of 3D, shaped form of a functional structure 100 that may have either a nonwoven or tissue substrate 200 and that contains SAP particles and/or fluff fiber as the functional material 400. The functional structures 100 prepared in Examples 6 and 7 can be rolled upon themselves to form tube-like (cylindrical) shaped forms and be used the same way as in Example 4.

### Example 9

The tube-like (cylindrical) shaped forms as prepared in Example 8 can be sliced into smaller length pieces and used as in Example 5.

### Example 10

Procedure for preparation of a 2D layer-by-layer (“L-B-L”) functional structure 100 using a soft template method. With a L-B-L functional structure 100 formation, several layers may be created over the substrate 100 using various deposition methods. Desired core wrap materials such as spunbond, SMS or tissue may be used as the substrate 200 if the functional structure 100 is to be used as a core wrap 740. Aqueous PVA solution or cross-linked PVA gel solution may be applied over the substrate 200 using dipping, spraying or coating. Desirably, the first fixing agent 300 should have limited solubility with water at room temperature in order to hold the functional material 400 (for example, SAP particles or fluff fiber) firmly during use. Additionally, the first fixing agent 300 needs to be hydrophilic and should not inhibit the absorbency of SAP particles or fluff fibers. Further, the PVA solution or cross-linked PVA gel solution desirably have limited solubility in water at room temperature and after application of SAP particles or fluff fiber in order to retain the original shape even after swelling of the SAP particles or fluff fiber. Next, functional material 400 is sprinkled over the first fixing agent 300. The layer of functional material 400 is porous in nature and quickly adheres to the first fixing agent 300. After application of the functional material 400, the second fixing agent 500 (for example, PVP solution) is spray applied under pressure. The second fixing agent 500 may cover the functional material 400 (SAP particles or fluff fibers) and may act as an intake system during use so that the functional structure 100 has good intake and absorbency.

### Example 11

Procedure for preparation of a 3D layer-by-layer (“L-B-L”) functional structure 100 using a soft template method. With a L-B-L functional structure 100 formation, several layers may be created over the substrate 100 using various deposition methods. Desired core wrap materials such as spunbond, SMS or tissue may be used as the substrate 200 if the functional structure 100 is to be used as a core wrap 740. A three-dimensional silicone sheet, acting as a template 600, is placed over the substrate 200. The template 600 may have various geometries or shapes and may help to preferentially distribute the other components of the functional structure 100. Aqueous PVA solution or cross-linked PVA gel solution may be applied over the substrate 200 using dipping, spraying or coating. Desirably, the first fixing agent 300 should have limited solubility with water at room temperature in order to hold the functional material 400 (for example, SAP particles or fluff fiber) firmly during use. Additionally, the first fixing agent 300 needs to be hydrophilic and should not inhibit the absorbency of SAP particles or fluff fibers. Further, the PVA solution or cross-linked PVA gel solution desirably have limited solubility in water at room temperature and after application of SAP particles or fluff fiber in order to retain the original shape even after swelling of the SAP particles or fluff fiber. Next, functional material 400 is sprinkled over the first fixing agent 300. The layer of functional material 400 is porous in nature and quickly adheres to the first fixing agent 300. After application of the functional material 400, the second fixing agent 500 (for example, PVP solution) is spray applied under pressure. The second fixing agent 500 may cover the functional material 400 (SAP particles or fluff fibers) and may act as an intake system during use so that the functional structure 100 has good intake and absorbency. After the functional structure 100 is dried, the template 600 may be removed.

In order to demonstrate the performance benefits of the shaped forms of functional structures 100 of the invention, disposable absorbent articles 700 including the following executions of shaped forms were tested using standard absorbency evaluation methods of intake and rewet as shown in Table 1 below.

TABLE 1

| | Intake | Intake | Intake | | | Total |
|---|---|---|---|---|---|---|
| | time-1 | time-2 | time-3 | Rewet-1 | Rewet-2 | Rewet |
| Examples | (seconds) | (seconds) | (seconds) | (grams) | (grams) | (grams) |
| Example 4, 6 gm SAM over | 13.40 | 19.8 | 15.64 | 0.67 | 1.56 | 2.23 |
| spunbond as substrate, | | | | | | |
| placed near bodyside liner | | | | | | |
| Example 4, 6 gm SAM over | 11.7 | 19.55 | 16.31 | 0.48 | 3.71 | 4.19 |
| tissue as substrate, placed | | | | | | |
| near bodyside liner | | | | | | |
| Example 5, 6 gm SAM | 16.95 | 26.51 | 45.49 | 0.22 | 0.26 | 0.48 |
| over spunbond as | | | | | | |
| substrate, placed near | | | | | | |
| bodyside liner | | | | | | |
| Example 8, 6 gm SAM | 13.98 | 20.84 | 30 | 0.15 | 0.18 | 0.33 |
| over spunbond as | | | | | | |
| substrate, placed near | | | | | | |
| bodyside liner | | | | | | |
| Control: core wrap with 28 | 19.59 | 14.7 | 24.06 | 0.16 | 0.1 | 0.26* |
| gsm of SAP particles over | | | | | | |
| fluff | | | | | | |
| Example 8, 6 gm SAM | 8.35 | 17.3 | 21.08 | 0.133 | 0.8 | 0.93 |
| over spunbond as | | | | | | |
| substrate, placed near | | | | | | |
| outer cover | | | | | | |
| Example 4, 6 gm SAM | 9.01 | 16.71 | 24.04 | 0.077 | 0.145 | 0.21 |
| over spunbond as | | | | | | |
| substrate, placed near | | | | | | |
| outer cover | | | | | | |
| Example 9, 6 gm SAM | 8.57 | 15.54 | 18.03 | 0.08 | 0.127 | 0.2 |
| over spunbond as | | | | | | |
| substrate, placed near | | | | | | |
| bodyside liner | | | | | | |
| Example 8, 3 gm SAM | 13.11 | 19.23 | 22.83 | 0.137 | 0.293 | 0.52 |
| over spunbond as | | | | | | |
| substrate, placed near | | | | | | |
| outer cover | | | | | | |
| Control: standard core wrap | 8.87 | 13.12 | 14.62 | 0.21 | 3.26 | 3.47 |

*With this “Control” example, the product post introduction of the fluid insults became very bulgy with free fluid. The product had an appearance of a water-filled bag that would be expected to have an overall negative consumer perception.

The results of Table 1. above show that disposable absorbent articles including the shaped forms of the invention perform well for fluid intake times and for fluid rewet (movement of fluid out of the absorbent core back out through the bodyside liner). The first, second and third intake times are for first, second and third insults of fluid applied to the samples.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. A disposable absorbent article comprising:
a bodyside liner;
an outer cover;
an absorbent core between the bodyside liner and the outer cover;
a core wrap surrounding the absorbent core; and
a shaped form between the core wrap and the absorbent core wherein the shaped form is a functional structure comprising a substrate; a first fixing agent applied to the substrate; wherein, the first fixing agent includes an aqueous polymer; a functional material adjacent the first fixing agent; and a second fixing agent adjacent to the functional material.

2. The disposable absorbent article of claim 1, wherein the first fixing agent is a hydrophilic solution including an aqueous polymer selected from polyvinyl alcohol and a cross-linked polyvinyl alcohol.

3. The disposable absorbent article of claim 2, wherein the aqueous polymers have molecular weights from 1,500,000 to 4,000,000.

4. The disposable absorbent article of claim 1, wherein the functional material is selected from superabsorbent material, pulp fiber material, deodorizing material and fragrance material.

5. The disposable absorbent article of claim 1, wherein the functional structure has a center and at least one outer edge and wherein the functional material has a higher concentration at the center of the functional structure than at the outer edge of the functional structure.

6. The disposable absorbent article of claim 1, wherein the functional material is present in a grid pattern of square-shaped areas of the functional material.

7. The disposable absorbent article of claim 1, wherein the second fixing agent is a hydrophilic solution of an aqueous polymer selected from polyvinylpyrrolidone in a C1 to C3 alcohol.

8. The disposable absorbent article of claim 7, wherein the aqueous polymer has a molecular weight of at least 30,000.

9. The disposable absorbent article of claim 1, wherein the shaped form is selected from a tube-like, cylindrical shape and an accordion-like, pleated shape.

10. The disposable absorbent article of claim 9, wherein the shaped form has a length from 0.3 centimeters to 30 centimeters.

11. A disposable absorbent article comprising:
a bodyside liner;
an outer cover;
an absorbent core between the bodyside liner and the outer cover;
a core wrap surrounding the absorbent core;
a surge layer between the bodyside liner and the core wrap; and
a shaped form between the surge layer and the core wrap wherein the shaped form is a functional structure comprising a substrate; a first fixing agent applied to the substrate;
wherein, the first fixing agent includes an aqueous polymer; a functional material adjacent the first fixing agent; and a second fixing agent adjacent to the functional material.

12. The disposable absorbent article of claim 11, wherein the first fixing agent is a hydrophilic solution including an aqueous polymer selected from polyvinyl alcohol and a cross-linked polyvinyl alcohol.

13. The disposable absorbent article of claim 12, wherein the aqueous polymers have molecular weights from 1,500,000 to 4,000,000.

14. The disposable absorbent article of claim 11, wherein the functional material is selected from superabsorbent material, pulp fiber material, deodorizing material and fragrance material.

15. The disposable absorbent article of claim 11, wherein the functional structure has a center and at least one outer edge and wherein the functional material has a higher concentration at the center of the functional structure than at the outer edge of the functional structure.

16. The disposable absorbent article of claim 11, wherein the functional material is present in a grid pattern of square-shaped areas of the functional material.

17. The disposable absorbent article of claim 11, wherein the second fixing agent is a hydrophilic solution of an aqueous polymer selected from polyvinylpyrrolidone in a C1 to C3 alcohol.

18. The disposable absorbent article of claim 17, wherein the aqueous polymer has a molecular weight of at least 30,000.

19. The disposable absorbent article of claim 10, wherein the shaped form is selected from a tube-like, cylindrical shape and an accordion-like, pleated shape.

20. The disposable absorbent article of claim 19, wherein the shaped form has a length from 0.3 centimeters to 30 centimeters.
